(19) United States
(12) Patent Application Publication
(10) Pub. No.: US 2023/0284906 A1
(43) Pub. Date: Sep. 14, 2023

(54) RISK STRATIFICATION METHOD FOR THE DETECTION OF CANCERS IN PRECANCEROUS TISSUES

(71) Applicant: The Curators of the University of Missouri, Columbia, MO (US)

(72) Inventors: Rong Wang, Leawood, KS (US); Yong Wang, Overland Park, KS (US)

(21) Appl. No.: 18/182,846

(22) Filed: Mar. 13, 2023

Related U.S. Application Data
(60) Provisional application No. 63/319,424, filed on Mar. 14, 2022.

(51) Int. Cl. A61B 5/00; G06T 7/00; G01N 21/35; G06V 20/69

(52) U.S. Cl. CPC A61B 5/0075; G06T 7/0012; G01N 21/35; G06V 20/695; G01N 2021/3595; G06T 2207/30024; G06T 2207/10056

(57) ABSTRACT

A method of stratifying precancerous tissues by their risk of becoming cancerous by using a machine learning algorithm in combination with hyperspectral imaging. Also a method of constructing the machine learning algorithm for stratifying precancerous tissues by risk.

### CROSS-REFERENCE TO RELATED APPLICATIONS

The present application claims priority benefits from U.S. provisional patent application Ser. No. 63/319,424 filed Mar. 14, 2022.

### FIELD

The present teachings relate to cancer, and more particularly to a risk classification strategy that can be utilized to detect cancers in their precancerous stages.

### BACKGROUND

The statements in this section merely provide background information related to the present disclosure and cannot constitute prior art.

Precancerous or premalignant lesions are abnormal bodily tissues associated with an increased risk of developing into cancers. A variety of organ systems are affected by precancerous lesions, including but not limited to the skin, mouth, cervix stomach, lungs, colon, and blood. In many cases, precancerous lesions will never become fully cancerous. Thus, clinicians cannot treat all precancerous lesions as likely cancers without incurring unacceptable waste in terms of money, time, and patient care. Nor can clinicians simply ignore precancerous lesions, however, as cancers are generally best treated earliest in their development. An objective clinical risk stratification of precancerous lesions by their likelihood to develop into cancer is therefore extremely desirable, both to properly treat patients who are likely to develop cancer and to avoid overtreatment of patients who will likely not develop cancer. Prevailing methods of precancer risk stratification using a traditional histopathological approach tend toward high subjectivity, low accuracy and large inter- and intra-observer variability among pathologists. The following disclosure details a novel method for accurately and objectively stratifying precancer according to their risk of becoming cancerous. Although such a method can pertain to a wide variety of bodily tissues, the following will provide exemplary and illustrative focus on oral cancers.

Oral cancer refers to a subgroup of head and neck malignancies that affect the lips, tongue, salivary glands, gingiva, floor of the mouth, buccal surfaces, and other intra-oral locations. It is one of the most prevalent cancers worldwide, with especially high incidence in low- and middle-income countries. Despite easy access to the oral cavity and new management strategies, oral cancer is still characterized by high morbidity and low survival rates, which are partially due to late diagnosis. More than 90% of oral cancers are oral squamous cell carcinoma (OSCC), which are a heterogeneous group of cancers arising from the mucosal lining of the oral cavity. Most oral cancer cases are associated with lifestyle habits including smoking, smokeless tobacco use, excessive alcohol consumption, and betel quid chewing. OSCC is 2-3 times more prevalent in men than it is in women, and its incidence is the highest in people who are older than 50 years of age. Genetic predisposition also plays an important role in the development of OSCC.

Oral carcinogenesis is a highly complex, multifactorial, and multistep process that can begin as hyperplasia/hyperkeratosis and can evolve to epithelial dysplasia, carcinoma in situ, and OSCC. Most OSCC are preceded by oral potentially malignant disorders (OPMDs), which are a heterogeneous group of clinical oral lesions (e.g., leukoplakia, erythroplakia, reverse smoker's palate, erosive lichen planus, oral submucous fibrosis, lupus erythematosus, and actinic keratosis) associated with a statistically increased risk of malignant transformation. OPMDs are common clinical lesions with an overall worldwide prevalence of 4.47%. They are visually detectable during routine dental examinations and present great opportunities for early oral cancer detection. To utilize this opportunity, accurate risk stratification for individual OPMDs is needed to identify patients most likely to develop a future OSCC. Unfortunately, the standard histopathology is incapable of doing that because it evaluates morphological changes of the tissue which don't always reflect the underlying pathological conditions. Therefore, there is an urgent need for a modern diagnostic tool that provides objective and accurate risk assessment of OPMDs for early oral cancer detection and prevention.

The clinical presentations of OPMDs can be further diagnosed as hyperplasia/hyperkeratosis (HK), oral epithelial dysplasia (OED), or OSCC via histopathological evaluation. Epithelial HK are a benign overgrowth of cells in the oral epithelium. They can represent the initial stage of cancer development. OED is defined as a precancerous lesion in the oral epithelial region where cells exhibit atypia up to a certain level of the epithelium. The diagnosis and grading of OED are mainly based on the combination of architectural changes and the appearance of specific histological features. An OED can be graded as mild, moderate, or severe based on a three-tier classification system developed by the World Health Organization (WHO). It has been estimated that 7-50% of severe, 3-30% of moderate, and <5% of mild OED lesions can transform into OSCC.

The gold standard WHO 2017 three-tier grading system for OED has some limitations, including subjectivity, inter- and intra-observer variations, and limited capability in predicting the malignant transformation risk of OED in individual cases. Suggestions to overcome these limitations include the use of clinical determinants and molecular markers to supplement the grading system. However, no single clinical-pathological predicting factor or molecular biomarker has achieved the clinical criteria for that purpose. Accurate risk assessment and the effective management of OPMD and OED play critical roles for improving oral cancer survival rates and prognosis. Therefore, there is a need for new biomarkers or modern techniques that can provide objective and accurate OPMD/OED risk stratification for early oral cancer detection and prevention.

### BRIEF SUMMARY

In various embodiments, presented herein is a method for stratifying precancerous tissues according to their risk of becoming cancerous. In various exemplary embodiments, the method uses the acquisition of hyperspectral images of tissue samples including benign tissue, one or more types of precancerous tissue, and cancerous tissue. Unsupervised exploratory analyses of hyperspectral images of tissue samples are then used to generate labeled hyperspectral images, which are then further analyzed according to one or more supervised discriminatory analyses. The supervised discriminatory analyses generate a discriminatory model that can determine the similitude of a subsequently acquired hyperspectral image of a tissue sample to the analyzed hyperspectral images corresponding to the benign tissue, one or more types of precancerous tissue, cancerous tissue. By determining which type of tissue a sample is most similar to, the discriminatory model can assign the sample to a corresponding risk stratum.

In various embodiments, the present disclosure provides a method for stratifying tissue samples into categories according to the similarity of their hyperspectral images to hyperspectral images of known categories of tissues, using unsupervised and supervised analyses, is also presented herein.

In various embodiments, the present disclosure provides a system for stratifying precancerous tissues in a bodily tissue sample by their risk of becoming cancerous, utilizing an FTIR microscope and a machine learning algorithm that is capable of recognizing a plurality of patterns of data and organizing the sources of those pluralities of data into corresponding categories. In various exemplary embodiments, the method utilizes the FTIR microscope to generate hyperspectral images of the precancerous tissues. The hyperspectral images comprise spectral data, a plurality of patterns of which are characteristic of the tissues from which the hyperspectral images have been acquired. The machine learning algorithm recognizes similar pluralities of patterns of data and uses these similarities to generate corresponding categories.

### BRIEF DESCRIPTION OF THE DRAWINGS

FIG. 1 is a diagrammatic outline of the process of developing a system for risk stratification of tissues, with exemplary and illustrative focus on oral tissues, in accordance with various embodiments of the present disclosure.

FIG. 2A provides an exemplary outline of the process of selecting areas of interest in tissue sections for hyperspectral imaging, in accordance with various embodiments of the present disclosure.

FIG. 2B Shows an exemplary depiction of a hyperspectral image generated from an area of interest, in accordance with various embodiments of the present disclosure.

FIG. 3A provides a diagrammatic exemplary outline of the process of preprocessing spectral data and performing unsupervised exploratory analyses for the purpose of constructing one or more discriminatory machine learning algorithm, in accordance with various embodiments of the present disclosure.

FIG. 3B provides an exemplary depiction of a hyperspectral image comprising a region of stromal tissue and a region of epithelial tissue. FIG. 3C provides an exemplary depiction of how unsupervised exploratory analyses such as hierarchical cluster analysis can distinguish infrared spectra corresponding to stromal tissue from infrared spectra corresponding to epithelial tissue.

FIG. 3D shows how data produced after the steps shown in FIG. 3A can be further analyzed to generate the one or more discriminatory machine learning algorithms, in accordance with various embodiments of the present disclosure.

FIG. 4A provides a diagrammatic outline of how the machine learning algorithm generated in the exemplary embodiment depicted in FIGS. 3A-3D can stratify the data processed according to process outlined in FIG. 3A by risk, in accordance with various embodiments of the present disclosure.

FIG. 4B provides a generalized outline of how one can use one or more discriminatory machine learning algorithms disclosed herein to stratify newly-sampled precancerous tissues by their risk of becoming cancerous, in accordance with various embodiments of the present disclosure.

FIG. 5 shows overlapping traces of averaged spectra for various types of precancerous oral tissue and indicates how subtle deviations in the amplitudes and shapes of particular spectral features are identifying of those types of precancerous oral tissue, in accordance with various embodiments of the present disclosure.

FIG. 6 provides an exemplary assignment table that links the average peak wavelength of particular spectral features with a vibrational mode that corresponds with each spectral feature, in accordance with various embodiments of the present disclosure.

FIG. 7A shows exemplary results of cross-validation for three different types of supervised discriminatory analyses as applied to an exemplary set of oral tissues, in accordance with various embodiments of the present disclosure.

FIG. 7B shows exemplary second-derivative spectra of latent variables derived from an exemplary PLSDA analysis of oral tissues to demonstrate what spectral features are emphasized by each latent variable, in accordance with various embodiments of the present disclosure.

FIG. 8 shows an exemplary depiction of a computer-based system as can be employed during operation of an FTIR microscope and/or during analysis of hyperspectral images.

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what we presently believe is the best mode of carrying out the claimed invention. Additionally, it is to be understood that the claimed invention is not limited in its applications to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

The term “OSCC” as used herein is an initialism that refers to oral squamous cell carcinoma.

The term “OPMD” as used herein is an initialism that refers to oral potentially malignant disorders.

The term “HK” as used herein is an initialism that refers to hyperkeratosis.

The term “OED” as used herein is an initialism that refers to oral epithelial dysplasia.

The term “WHO” as used herein is an initialism that refers to the World Health Organization.

The term “PCA” as used herein is an initialism that refers to principal components analysis, a statistical technique for reducing the dimensionality of a dataset.

The term “HCA” as used herein is an initialism that refers to hierarchical cluster analysis, a method of grouping data into clusters, or groups whose peers are more similar to one another than to data in other groups, while building a hierarchy of those clusters.

The term “unsupervised” as used herein refers to algorithms and techniques that analyze and organized entirely or substantially unlabeled data sets.

The term “supervised” used herein refer to algorithms and techniques designed to train a model to yield a desired output using typically labeled data sets.

The term “PLSDA” as used herein is an initialism that refers to partial least squares discriminant analysis, a supervised statistical method used to find fundamental relations between two matrices.

The term “SVMDA” as used herein refers to support vector machines discriminant analysis, a supervised linear statistical classification method.

The term “XGBDA” as used herein refers to extreme gradient boosting discriminant analysis, a supervised algorithm suited for non-linear parameters.

The term “ROC curve” as used herein refers to a receiver operating characteristic curve, which shows the performance of a classification model at all classification thresholds.

The terms “image” as used herein refer to photographs, spectral data, and any and all information acquired from the interaction of light of any frequency with a sample.

The term “imaging” as used herein refers to any means of acquiring an image.

The term “hyperspectral” as used herein describes an image that is constructed with the goal of obtaining a spectrum for each pixel in the image. Thus, a hyperspectral image is multidimensional, and unlike an image that conveys information from light acquired solely in the visual spectrum, can convey a broader variety of spectral information.

The term “risk” as used herein refers to the likelihood that a precancerous tissue will develop into a cancerous tissue.

The precancerous tissue risk stratification method disclosed herein requires the analysis of tissue samples via hyperspectral imaging. Hyperspectral images contain spatial data infrared light spectra that are processed and then analyzed to create machine learning algorithms that are constructed expressly for analysis of precancerous tissue sample images. Thus, the following disclosure will provide exemplary methods of precancerous tissue sampling and hyperspectral imaging used for precancerous tissue risk stratification. This disclosure will also provide methods of construction of the described machine learning algorithms used for precancerous tissue risk stratification, which are a core innovation of this method. Once these machine learning algorithms are constructed, one can use them to analyze ‘new’ precancerous tissues samples and thereby stratify the samples by risk of becoming cancerous. As will become clear, the tissue sample analysis method itself generates the tools required to operate the method at full competence to stratify precancerous tissues by their risk of becoming cancerous.

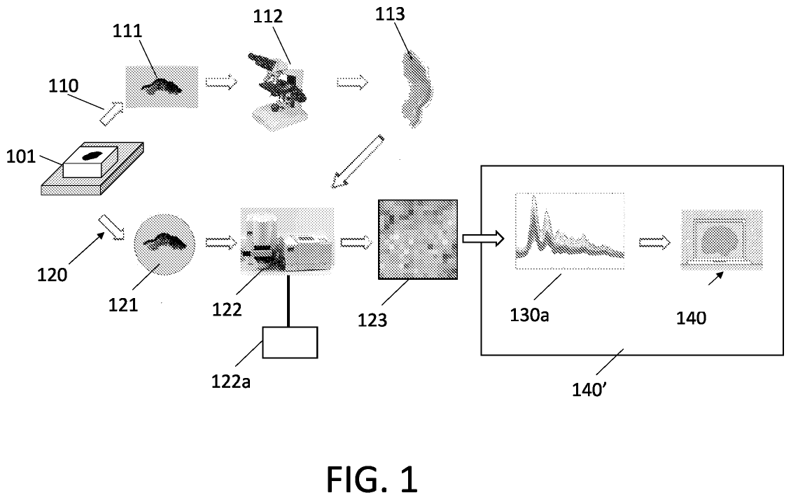

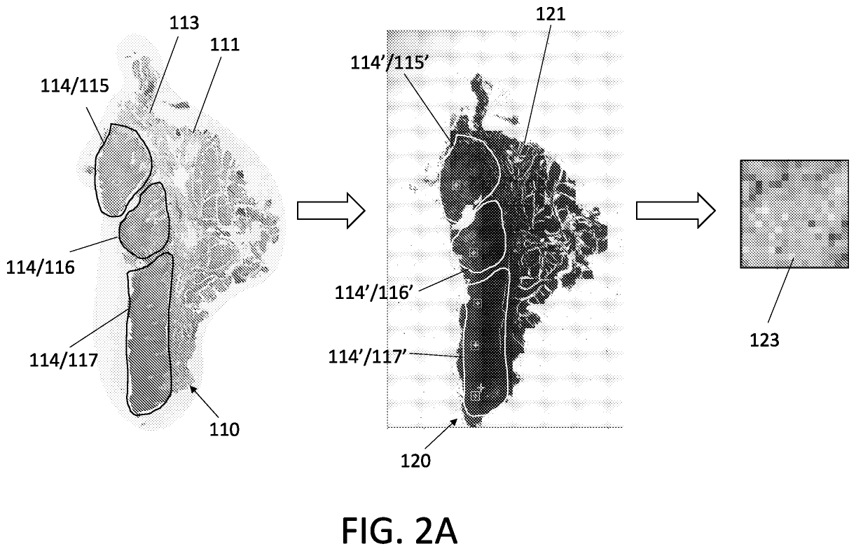

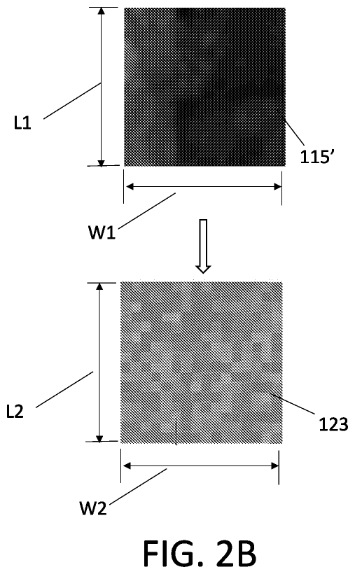

Referring to FIGS. 1, 2A and 2B, FIG. 1 exemplarily illustrates a process of developing a system for risk stratification of tissues in full, showing how spectroscopic analyses of precancerous tissue can be used in generating the disclosed machine learning algorithms. First, a biopsy is performed on a patient to produce a tissue sample 101. In various instances the tissue sample 101 can be an oral squamous cell carcinoma (OSCC) tissue (or any other tissue of concern) and can comprise one or more hyperkeratotic (HK) regions, one or more oral epithelial dysplastic (OED) regions, one or more OSCC regions, or any combination of HK, OED, and OSCC regions. HK regions contain a plurality of cells that that, in epithelial tissues, are benign overgrowths of tissue. OED regions contain a plurality of cells exhibiting atypia (unusual cellular or architectural features) oral epithelial cells displaying dysplasia are considered to be precancerous lesions. OSCC regions contain a plurality of cancerous cells. The tissue sample 101 can be formalin-fixed and paraffin-embedded (FPPE), but means of tissue preservation vary, so any effective means are within the scope of this disclosure.

From there, the tissue sample 101 is sectioned into at least a first section 110 (e.g, a 4-5 μm thick slice) and a second section 120 (e.g., a 4-5 μm thick slice). The first section 110 and second section 120 can, in various embodiments, be adjacent sections (e.g., adjacent thin slices) of the tissue sample 101 such that first section 110 and second section 120 are substantially identical, which aids direct comparison of the two sections. The first section 110 is then prepared for histopathological evaluation, which in various embodiments can comprise exposing the sample to a dye and thereby preparing a dyed sample 111. In various embodiments, the dyed sample 111 can be dyed with hematoxylin and eosin (H&E) or any other dye or tissue stain known to aid in the visual differentiation of cell and biological matrix components. The dyed sample 111 can then undergo optical microscopy (via an optical microscope) so that microscopic images 112 of the dyed sample can be generated. The microscopic images 112 then undergo evaluation by a histopathologist or other qualified entity or a computer-based histopathological analysis algorithm/program/software to select areas of the tissue images that show abnormalities or other signs indicative of precancerous lesions. The histopathological evaluation can result in the microscopic images 112 being annotated, referred to herein as annotated images 113, which, as described below, can aid in hyperspectral imaging.

Meanwhile, the second section 120 is prepared for Fourier transform infrared spectroscopy (FTIR) imaging, which captures spatially resolved FTIR spectra. FTIR spectroscopy is a technique that uses infrared light to probe the vibrational modes of chemical or biological analytes, thereby producing spectra that read as biochemical ‘fingerprints’ of the analytes. FTIR imaging is a type of hyperspectral imaging wherein each pixel of a hyperspectral image contains a full FTIR spectrum. To perform FTIR imaging, the second section 120 is applied to an optical substrate 121 that is transparent in a predetermined infrared (IR) frequency window, which, in various embodiments, can be anywhere from 4000 cm⁻¹ To 600 cm⁻¹, for example 1800 cm⁻¹ to 900 cm⁻¹. In various embodiments, the optical substrate can be a disc of barium fluoride (BaF₂), calcium fluoride (CaF₂), fused silica, or any other material known in the art to serve as an optical transmission window in the predetermined frequency range. If the sample 101 was initially preserved as with FFPE, then the preservative is removed to ensure that it does not interfere with FTIR spectroscopy.

In various exemplary embodiments, FFPE samples are deparaffinized through immersion in histological grade xylene, each for five minutes, at room temperature, after which point they are air dried and stored in a vacuum desiccator to remove as much residual moisture as possible. However, other means of removal of a preservative can be warranted depending on the means of preservation, and all are within the scope of the present disclosure. Additionally, in lieu of removing preservative, in various exemplary embodiments, the preservative's contribution to any acquired spectra can be removed, as by background subtraction.

Once prepared as described above, the second sample section 120 disposed on the optical substrate 121 is placed in suitable FTIR microscope 122. The FTIR microscope 122 is capable of acquiring multidimensional images of the second sample section 120. However, it is often impractical to image the entire second section 120, so in various embodiments, as exemplarily illustrated in FIG. 2A, since the first and second sections 110 and 120 are substantially identical, annotated images 113 resulting from the histopathological evaluation of the first section 110 of the tissue sample 101 are used to refine what areas of the second section 120 of the tissue sample 101 will be imaged to produce one or more hyperspectral images 123 with the FTIR microscope.

This process is depicted in greater detail in an exemplary embodiment in FIG. 2A, wherein an exemplary annotated image 113 of a first section 111 is shown resulting from the histopathological analysis. One or more regions of the annotated image 113 are annotated to denote one or more areas of interest (AOI) 114. In the exemplary embodiment depicted in FIG. 2A, the annotated image 113 comprises three AOI 114: an HK region 115, an OED region 116, and an OSCC region 117. These AOI 114 as shown in FIG. 2A are purely exemplary and are not meant to depict features or elements characteristic of such regions. Actual tissue samples will vary, however, and can contain only one such AOI 114 or some combination of the three. In various embodiments, the annotated image 113 can comprise one AOI 114 or a plurality of AOIs. In various embodiments, one or more AOI 114 can be selected such that, if it/they contain one or more HK region 115, the HK region(s) 115 primarily comprise epithelial tissue. In various embodiments, one or more AOI 114 can be selected such that, if it/they contain one or more OED region 116, the OED region(s) 116 primarily comprise epithelial tissue. In various embodiments, one or more AOI can be selected such that, if it/they contain one or more OSCC region 117, the OSCC region(s) 117 can comprise primary cancerous and/or invasive cancerous regions, where an invasive cancerous region comprises a cancer that has spread beyond the layer of tissue in which it initially developed. In various embodiments, one or more AOI 114 can be chosen to exclude tissues that visibly have poor structural integrity to ensure high-quality hyperspectral imaging.

As noted above, in various exemplary embodiments, the first section 110 and the second section 120 of the tissue sample 101 are thin adjacent sections and, as a result, are almost identical in composition. Therefore, the one or more AOI 114 identified in first section 110 during histopathological analysis correspond to one or more AOI 114′ of substantially the same composition in the second section 120. Furthermore, the HK regions 115, OED regions 116, and OSCC regions 117 in the first section 110 correspond to complementary HK regions 115′, OED regions 116′, and OSCC regions 117′ in the second section. The second section 120 is placed in an FTIR microscope 122. In various embodiments, the FTIR microscope 122 is operably connected to a computer-based system 122a that is structured and operable to receive inputs (e.g., image data) from the FTIR microscope and any other system or device described and/or illustrated herein and execute various software and/or algorithms to analyze the received data and calculate risk stratification of selected tissue samples as described and illustrated throughout the present disclosure. The FTIR microscope 122 is used to acquire visual survey images of the second section 120, in part or in whole, and spectral data from the one or more HK regions 115′, OED regions 116′, and/or OSCC regions 117′, resulting in the generation of one or more hyperspectral images 123. By acquiring hyperspectral images from the second section 120 instead of the first section 110, any dyes or other visual histopathological aids applied to first section 110 will not interfere with the FTIR microscope 122. The one or more hyperspectral images 123 are then organized by the type of cancerous or precancerous tissue images that they reveal. Generally, background correction can comprise acquiring an image of the clean optical substrate 121 that can subsequently be subtracted from future imaging spectra as a means of background correction.

Although the exemplary embodiment depicted in FIG. 1 and FIG. 2A uses the one or more AOI 114 identified a first section 110 to guide the use of the FTIR microscope 122 in acquiring hyperspectral images 123 of one or more AOI 114′ in the second section 120, ordinary variations evident to one of ordinary skill are within the scope of the present disclosure. For example, in various embodiments, the optical functionality of the FTIR microscope 122 in the visual spectrum can be used to identify one or more AOI in the second section without any regard to analysis of the first section 110.

FIG. 2B provides a depiction of an exemplary image acquisition sequence focusing on a selected OED region 116. In this exemplary depiction, a 100 μm×100 μm subset 115′ of the HK region 115 is chosen as an imaging area of the tissue sample second section 120. The subset 115′ has a width W1 and a length L1. In the exemplary embodiment in FIG. 2B, width W1 is 100 μm and length L1 is 100 μm. In various exemplary embodiments, the width W1 and length L1 can be any value as defined by the limits of the FTIR imaging instrument used and the needs and interests of the operator. FTIR image acquisition is performed on this area, resulting in the pixelated hyperspectral image 123. The hyperspectral image 123 has a width W2 and a length L2. The hyperspectral image 123 shown in the exemplary depiction of FIG. 2B has a width W2 of 16 pixels and a length L2 of 16 pixels, but the width and length in pixels of the one or more hyperspectral images generated by FTIR imaging of an imaging area of any given size will depend on the resolution and operating parameters of the FTIR imaging instrument. As the data in each pixel also comprises an FTIR spectrum, higher-resolution one or more hyperspectral images 123 will scale in total data size very rapidly. As shown in FIG. 1, data from the hyperspectral image 123 is then collated into a group of all raw spectra 130a. These raw spectra 130a are then used to construct the set of machine learning algorithms 140. In various exemplary embodiments, the collation into a group of all raw spectra 130a as well as the subsequent construction of machine learning algorithms 140 occurs via a computer-based control system 140′.

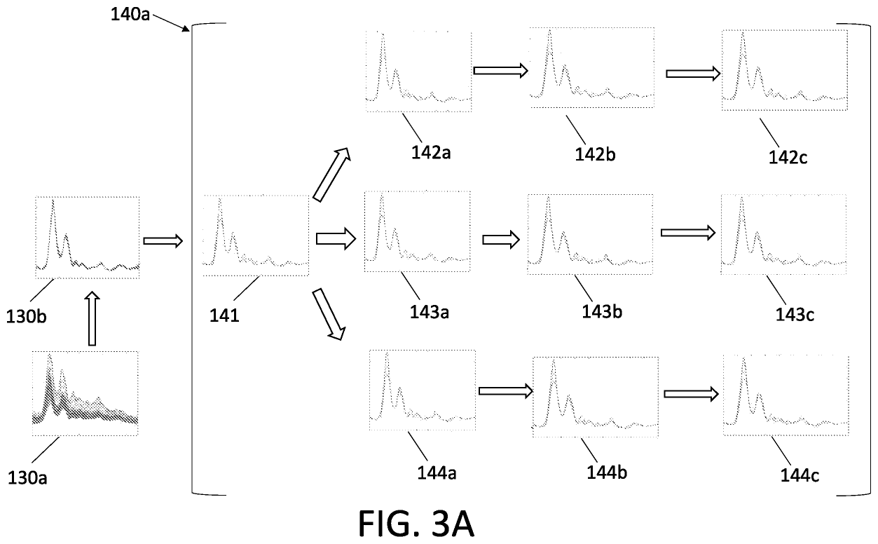

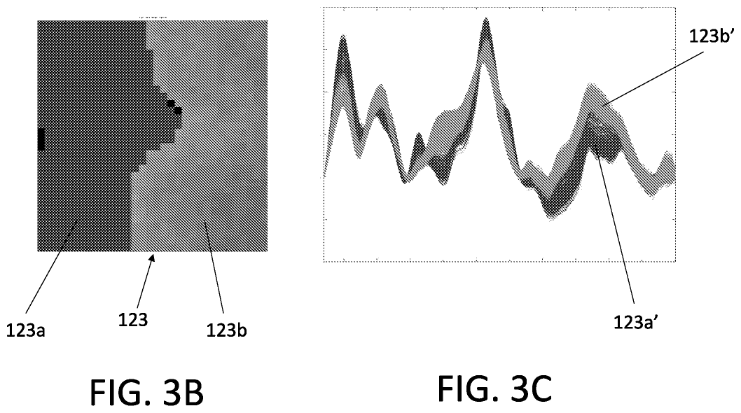

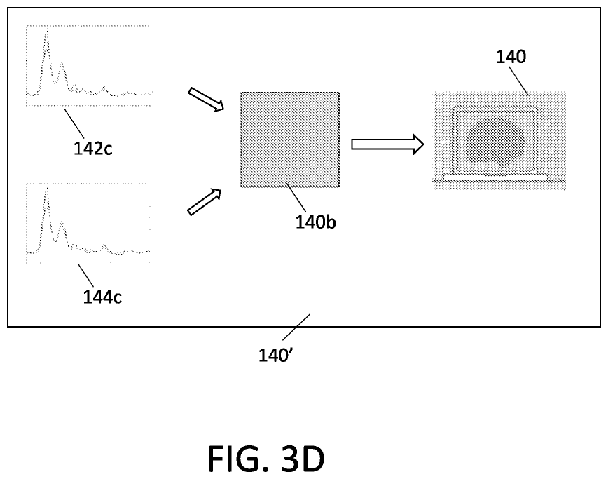

FIG. 3A depicts in detail how the raw spectra 130a are used to construct the set of machine learning algorithms 140 in various exemplary embodiments. First, the set of raw spectra 130a undergo preprocessing to become preprocessed spectra 130b. Preprocessing describes the use of known techniques to clarify the relevant signals in spectral data by reducing or eliminating spectral features originating from various environmental and structural elements that are not germane to the sample analysis. In various embodiments, preprocessing proceeds through a six-step process comprising a transmission/absorbance conversion, a selection of a fingerprint region, a digital filtering, a light-scattering correction, a baseline correction, and a normalization. In various embodiments, the transmission/absorbance conversion can use known equations that convert between absorbance and transmission values, resulting in data of whichever form is desired. In various embodiments, the selection of a fingerprint region requires choosing a frequency region in which relevant spectral data is located, thereby excluding data from other frequencies. In at least one exemplary embodiment, the fingerprint region was selected as 1800-950 cm⁻¹.

In various embodiments, the digital filtering step smooths data by convolution to suppress or eliminate the contributions of noise. In at least one exemplary embodiment, the digital filtering step can be performed by applying a Savitsky-Golay filter. In various embodiments, the light scattering correction can be performed by known technique to reduce or eliminate the features and effects in spectra that are contributed by physical phenomena such as scattering rather than the vibrational, rotational, and other chemical resonance phenomena intentionally probed by spectroscopy. In at least one exemplary embodiment, the light scattering correction can be extended multiplicative scattering correction (EMSC). In various embodiments, baseline correction can be applied in order to reduce or eliminate apparent artificial contributions to the signal that are caused by baseline variations created during background subtraction. In at least one exemplary embodiment, the baseline correction can be automated weighted least squares (AWLS) baseline correction. In various embodiments, vector normalization can be performed by any known means, and permits the more accurate cross comparison of spectra by normalizing spectra to minimize errors resulting from effects such as variable sample thickness. In various embodiments and as shown in FIG. 1, all preprocessing is applied/performed through the use of a computer-based system 140′.

Although in various exemplary embodiments preprocessing occurs through a six-step process as outline above, variations in the number and type of preprocessing steps evident to those of ordinary skill in the art are considered to be within the scope of the present disclosure.

Once preprocessing is complete, the preprocessed spectra 130b are then used to construct, and are in turn interpreted by, machine learning algorithms 140. Turning to FIGS. 3A-3D, the machine learning algorithms 140 comprise both unsupervised exploratory analyses 140a and supervised discriminatory analyses 140b, and in various embodiments, the particular analyses and the order in which they proceed can vary. FIG. 3A presents an exemplary embodiment that describes one possible order in which machine learning algorithms 140 can proceed with unsupervised exploratory analyses 140a. First, the preprocessed spectra 130b undergo unsupervised exploratory analyses, resulting in refined spectra 141. Unsupervised exploratory analyses are, broadly, mathematical and statistical analyses performed for a variety of reasons, including understanding how variables in a data set relate to each other and how samples in which those variables are studied relate to each other. In various embodiments, the unsupervised exploratory analyses are performed in order to eliminate outliers and ensure that spectra are only representative of cells of interest from the regions 115, 116, and 117. For example, in various embodiments, unsupervised exploratory analyses can comprise one or more distinct analyses including Principal Components Analysis (PCA) and Hierarchical Cluster Analysis (HCA). PCA is a known method used to reduce the number of dimensions in large data sets by transforming the data set into a new coordinate system that describes the data according to ‘principal components’ which best explain variance in the data.

In various exemplary embodiments in which PCA is performed during unsupervised exploratory analyses, it results in the identification of key spectral features as variables that distinguish between spectra from different groupings. This can help to organize the data and to identify outlier spectra. HCA works by organizing data into clusters based on the mutual similarities and variances in the data, and then organizing those clusters into hierarchical levels. In the various exemplary embodiments in which cluster analysis is performed, it enables the separation of spectra corresponding to one cell type from those of another cell type; for example, in various embodiments HCA can separate epithelial cell spectra from nonepithelial cell spectra. This is broadly useful as a method of more finely separating tissues by cell type after FTIR image acquisition has taken place.

Refined spectra 141 are then stratified according to the region of tissue from which the spectra were acquired. Thus, spectra acquired from HK regions 115 are stratified into a group of refined HK spectra 142a, spectra acquired from OED regions 116 are stratified into a group of refined OED spectra 143a, and spectra acquired from OSCC regions 117 are stratified into a group of refined OSCC spectra 144a.

In various exemplary embodiments, each set of refined spectra 142a, 143a, and 144a are viewed and evaluated for quality. This scrutiny results in the selection of subsets of high-quality spectra. Thus, scrutiny and selection of the best spectra from the refined HK spectra 142a results in representative HK spectra 142b, while the same process applied to refined OED spectra 143a results in representative OED spectra 143b, and the same process applied to refined OSCC spectra 144a results in representative spectra 144b.

In various exemplary embodiments, each of the sets of representative spectra 142b, 143b, and 144b undergo further unsupervised exploratory analysis as described previously to further identifies trends, patterns, and groupings in each set of spectra. The use of unsupervised exploratory analyses on the representative HK spectra 142b, OED spectra 143b, and OSCC spectra 144b result in explored HK spectra 142c, explored OED spectra 143c, and explored OSCC spectra 144c respectively.

In various exemplary embodiments, unsupervised exploratory analyses including but not limited to HCA can be used to identify different categories of tissues by their distinct spectra. Turning to FIGS. 3B-3C, an exemplary hyperspectral image 123 comprises a stromal tissue region 123a and an epithelial tissue region 123b. Unsupervised exploratory analyses 140a such as HCA can be used to distinguish the infrared spectral features of stromal tissues 123a′ from the infrared spectral features of epithelial tissues 123b′.

Turning to FIG. 3D, the explored HK spectra 142c and explored OSCC spectra 144c are used to construct discriminant machine learning models 140b via the use of supervised learning which, in various embodiments, is performed by the computer-based system 140′. Supervised learning, broadly, refers to a strategy of analyzing labeled data to generate one or more functions or algorithms that reliably map aspects of that data to the data labels. For example, supervised learning as applied to explored HK spectra 142c and explored OSCC spectra 144c can comprise the generation of one or more functions or algorithms that accurately and reliably map variables in the spectral data to HK or OSCC cell types. The goal of such a strategy is to be able to later analyze unlabeled spectra (that is, spectra that have not been previously labeled as being acquired from HK tissues or OSCC tissues) and, from that spectral data, inductively infer which spectra correspond to HK tissues and which correspond to OSCC tissues.

In various exemplary embodiments, supervised learning can comprise supervised algorithms such as “partial least squares discriminant analysis” (PLSDA), “support vector machines discriminant analysis” (SVMDA), and “extreme gradient boosting discriminant analysis (XGBDA). PLSDA is a known method for classifying spectral data that works well when used with a small sample set that has data with a large number of variables and a high degree of correlation between variables. However, PLSDA performance can degrade when nonlinearity is present in data that it analyzes. SVDMA is also a known method that excels when used with sample sets that have a large number of variables, and it is robust against a degree of nonlinearity that can inhere in the data that it analyzes. XGBDA is even more robust against data that exhibits nonlinearity and outliers but has been observed to overfit the data.

In the exemplary embodiment depicted in FIG. 3A, a particular sequence of preprocessing and unexplored analysis steps is described, but in various alternative embodiments a different set of steps can be followed. For example, in various alternative embodiments, other known techniques for dimensionality reduction such as non-negative matrix factorization (NMF) and independent component analysis (ICA) can be used. Furthermore, in various alternative embodiments, other means of eliminating outliers can be used, including visual assessment by an operator of the method.

Turning to FIG. 3D, the explored HK spectra 142c and explored OSCC spectra 144c are analyzed by one or more supervised algorithms 140b, in this exemplary embodiment PLSDA, to construct the machine learning algorithm 140. The machine learning algorithm 140 is thus trained on high-quality labeled HK and OSCC spectra 142c and 144c and is able to distinguish HK spectra 142c from OSCC spectra 144c. In various embodiments, the construction of the machine learning algorithm 140 is an object of the present disclosure.

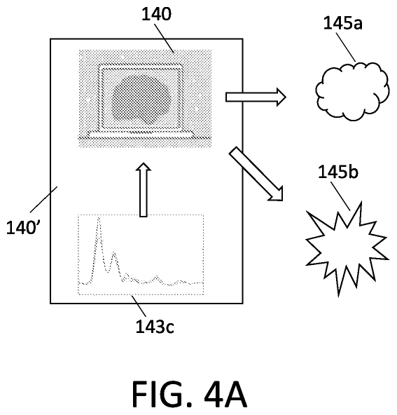

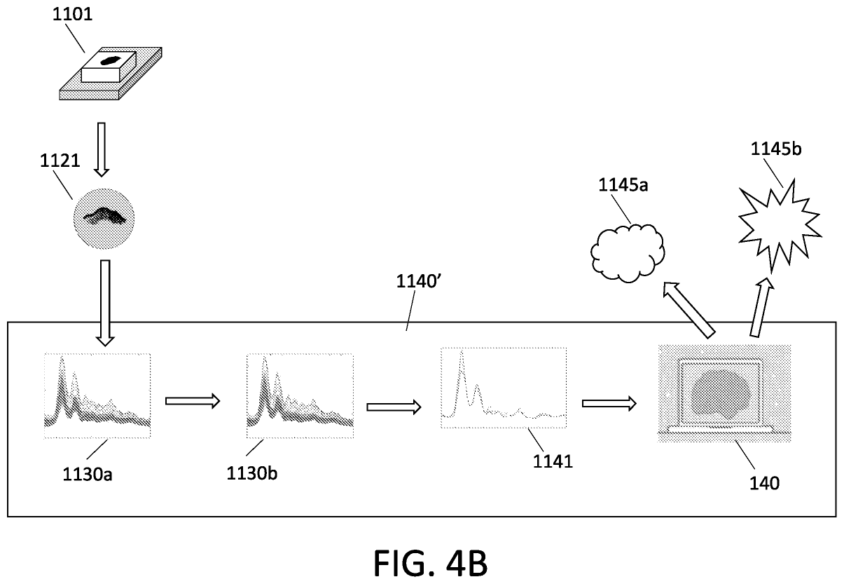

Once the machine learning algorithm 140 has been created, it can be implemented to analyze hyperspectral images of OED tissues to stratify those tissues by their risk of becoming cancerous. Turning to FIG. 4A, in various exemplary embodiments, explored OED spectra 143c are fed into the machine learning algorithm 140 through the use of the computer-based system 140′. The OED tissues represented in the explored OED spectra 143c, by their nature, are characterized by cytological and architectural abnormalities, but the rate at which they actually become cancerous can vary from 3% to 50%. Thus, the goal in the exemplary embodiment depicted in FIG. 4A is to have the machine learning algorithm 140 analyze the explored OED spectra 143c to determine how the machine learning algorithm labels these spectra. Since the machine learning algorithm has been trained to recognize subtle patterns in spectral data distinguishing more benign HK cells from fully cancerous OSCC cells, the machine learning algorithm 140 is capable of recognizing those same patterns, where they are most relevant, in OED cell spectral data. The result of analyzing explored OED spectra 143c with the machine learning algorithm 140 is, therefore, a determination as to whether the tissues of the OED region 116 from which those spectra were acquired belong to a lower risk stratum 145a, described by being more similar to HK cells, or to a higher risk stratum 145b, described as being more similar to cancerous OSCC cells. This risk objective, data-driven risk stratification is an object of the present disclosure.

In the exemplary embodiment depicted in FIG. 4A, explored OED spectra 143c were the result of hyperspectral imaging of histopathologically relevant tissue regions, wherein the resulting spectra underwent unsupervised exploratory analyses 140a. However, in various alternative embodiments, the composition, order, and extent of preprocessing and unsupervised exploratory analysis 140a steps can vary significantly depending on the quality of the tissue sample second section 120, the quality of the spectral images 123, and the needs of a scientific or medical professional. For example, multiple representative spectra acquired from the area of interest can be averaged before being analyzed by machine learning algorithm 140. Spectra acquired from the area of interest can only comprise cells of one or two types, such as OED cells.

One such alternative embodiment is depicted exemplarily in FIG. 4B. In this alternative exemplary embodiment, a new tissue sample 1101 is acquired by patient biopsy or any other means as previously described. The new tissue sample 1101 is sectioned to produce a tissue section 1121. Potential areas of interest in the tissue section 1121 are identified and then spectrally imaged as described above with regard to FIGS. 2A-2B, generating spectra of interest 1130a. The proper labeling of tissue types represented in spectra of interest 1130a can be unknown, and thus the spectra of interest can comprise spectra acquired from HK cells, OED cells, OSCC cells, or some mixture thereof. Spectra of interest 1130a then undergo preprocessing via the aid of computer-based system 1140′ to generate preprocessed spectra 1130b. The preprocessed spectra 1130b undergo unsupervised exploratory analysis as described above to generate refined spectra 1141. These refined spectra 1411 are analyzed by the machine learning algorithm 140, resulting in each analyzed spectrum being labeled as belonging either to a lower risk stratum 1145a or a higher risk stratum 1145b.

In various exemplary embodiments, spectra from various tissues can undergo further preprocessing before being analyzed via supervised discriminatory analyses 140b. For example, the first derivative, second derivative, or a higher-power derivative of spectra from hyperspectral images can be calculated, and these derivative spectra can be analyzed by supervised discriminatory analyses 140b. All additional preprocessing known to one of ordinary skill in the art is within the scope of the present disclosure.

In various exemplary embodiments, the stratification method of the present disclosure can be augmented by an image-based classifier using a deep learning image recognition and classification system such as a convolutional neural network (CNN). In various exemplary embodiments, the CNN can be used to for finding patterns in the one or more hyperspectral images 123, leveraging both the spectral and spatial information in each hyperspectral image for more comprehensive, accurate, and biologically meaningful classifications. In various exemplary embodiments, the outputs of multiple individual discriminant analyses, including but not limited to CNN and PLSDA, can be used as inputs to train a machine learning meta-classifier that can generate a final precancerous tissue risk stratification result.

In various exemplary embodiments, all control and operation of the FTIR microscope 122, preprocessing, unsupervised exploratory analyses 140a, and supervised discriminatory analyses 140b can occur with the aid of one or more of the computer-based systems 122a, 140′, and 1440′. Although the exemplary embodiments described herein provided for at least two separate computer-based systems for operation of the FTIR microscope 122a and machine learning algorithm 140, in various embodiments, any number of computer-based systems can be used according to the needs and convenience of the operator.

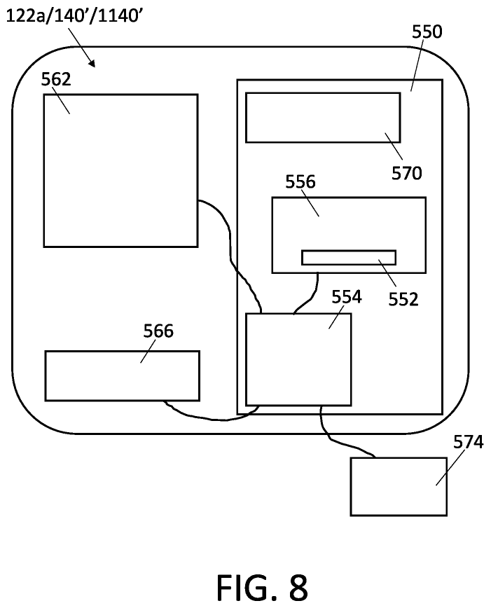

In various exemplary embodiments, the computer-based systems 122a, 140′, and 1140′ can be as shown and described as exemplarily depicted in FIG. 8. Referring to FIG. 8, the computer-based systems 122a, 140′, and 1440′ includes various computers, controllers, programmable circuitry, electrical modules, etc. that can be located at various locations with respect to the FTIR microscope 122. Particularly, in various embodiments, the computer-based systems 122a, 140′, and 1140′ can include one or more computers and/or computer-based modules 550 that each include at least one processor 554 suitable to execute the various software, programs, algorithms, and/or code that control all automated functions, operations, and analyses of the FTIR microscope 122 and/or any data analytics suites amenable to preprocessing, unsupervised exploratory analyses 140a, and/or supervised discriminatory analyses 140b. Each computer and/or computer-based module 550 can additionally include at least one electronic storage device 556 that comprises a computer readable medium, e.g., non-transitory, tangible, computer-readable medium, such as a hard drive, erasable programmable read-only memory (EPROM), electronically erasable programmable read-only memory (EEPROM), read-write memory (RWM), etc. Other, non-limiting examples of the non-transitory, tangible, computer-readable medium are nonvolatile memory, magnetic storage, and optical storage. Generally, the computer readable memory can be any electronic data storage device for storing such things the various software, programs, algorithms, code, digital information, data look-up tables, spreadsheets and/or databases, etc., used and executed during operation of the FTIR microscope 122 or any software used during preprocessing or supervised 140a or unsupervised 140b analyses of data, as described herein.

Furthermore, in various implementations, the computer-based system 122a/140′/1440′ can include at least one display 562 for displaying such things as information, data and/or graphical representations, and at least one user interface device 566, such as a keyboard, mouse stylus, and/or an interactive touch-screen on the display 566. In various embodiments, some or all of the computers and/or computer-based modules 550 can include a removable media reader 570 for reading information and data from and/or writing information and data to removable electronic storage media such as floppy disks, compact disks, DVD disks, zip disks, flash drives or any other computer-readable removable and portable electronic storage media. In various embodiments the removable media reader 570 can be an I/O port of the respective computer or computer-based module 550 utilized to read and/or receive data from external devices such as the FTIR microscope 122 or peripheral memory devices such as flash drives or external hard drives.

In various embodiments, the computer-based system 122a/140′/1440′, e.g., one or more of the computers and/or computer-based modules 550, can be communicatively connectable to a remote server network 574, e.g., a local area network (LAN), via a wired or wireless link. Accordingly, the computer-based system 530 can communicate with the remote server network 574 to upload and/or download data, information, algorithms, software programs, and/or receive operational commands. Additionally, in various embodiments, the computer-based system 530 can be constructed and operable to access the Internet to upload and/or download data, information, algorithms, software programs, etc., to and from Internet sites and network servers. In various embodiments, the various FTIR microscope and data analytics software, programs, algorithms, and/or code executed by the processor(s) 354 to control the operations of the FTIR microscope and/or data preprocessing, unsupervised analysis 140a, and/or supervised analysis 140b can be top-level system control software that not only controls discrete hardware functionality, but also prompts an operator for various inputs.

Although the disclosure provided herein has placed exemplary and illustrative focus on the stratification of OED tissues, the method herein disclosed can be applied to risk stratification of tissues featuring oral potentially malignant disorders (OPMD) generally. Thus, in stratification of the risk of a precancerous oral tissue becoming cancerous according to the present method, one can acquire and analyze hyperspectral images of oral tissues that do not necessarily display oral epithelial dysplasia specifically but belong to a category of OPMD tissues.

Although the disclosure provided herein has placed exemplary and illustrative focus on oral cancers, the method herein disclosed can be applied to risk stratification of other precancerous tissues as well. For example, in various embodiments, the method disclosed herein can be used to stratify cervical tissues by risk of becoming cancerous. Precancerous cervical epithelial cells are typically histologically graded into at least three strata. Thus, application of the herein disclosed method to cervical cells would comprise the generation of machine learning algorithms through the unsupervised exploratory analyses and supervised discriminatory analyses of spectra from cells from each histological grade as well as fully cancerous cervical cells. Once constructed, such machine learning algorithms can then analyze other precancerous tissue samples to classify them into one of a plurality of risk strata. Thus, not only can the described method apply to a plurality of types of precancerous tissues, but can assign tissues to a plurality of risk strata, not necessarily just two strata as in the exemplary embodiments described with respect to oral precancerous tissues.

### Examples

The following examples comprise descriptions of exemplary embodiments of the herein discloses method of analysis. These examples are not intended to be limiting or to define the scope of the present disclosure.

Comparison of Class-Average Spectra

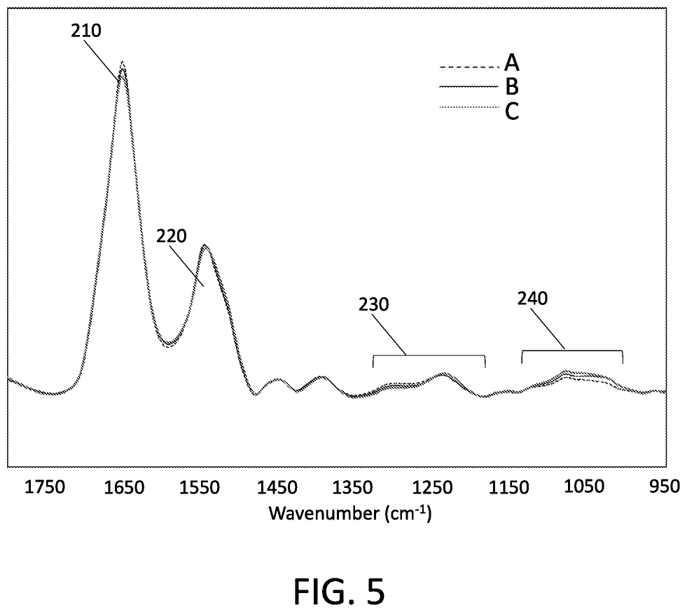

An exemplary execution of the herein disclosed method was performed to create a machine learning algorithm for the risk stratification of precancerous oral tissues. In this exemplary execution, as shown in FIG. 5, representative spectra from each cell type, HK, OED, and OSCC, are averaged to produce an average HK spectrum (trace ‘A’), an average OED spectrum (trace ‘B’), and an average OSCC spectrum (trace ‘C’). These averaged spectra are overlapped to show subtle differences in the shape and amplitude of certain spectral features. These spectral features are the Amide I band 210 at approximately 1650 cm⁻¹, the Amide II band 220 between approximately 1600 and 1500 cm⁻¹, the Amide III band 230 between approximately 1350 and 1180 cm⁻¹, and the glycogen band 240 between approximately 1160 and 950 cm⁻¹.

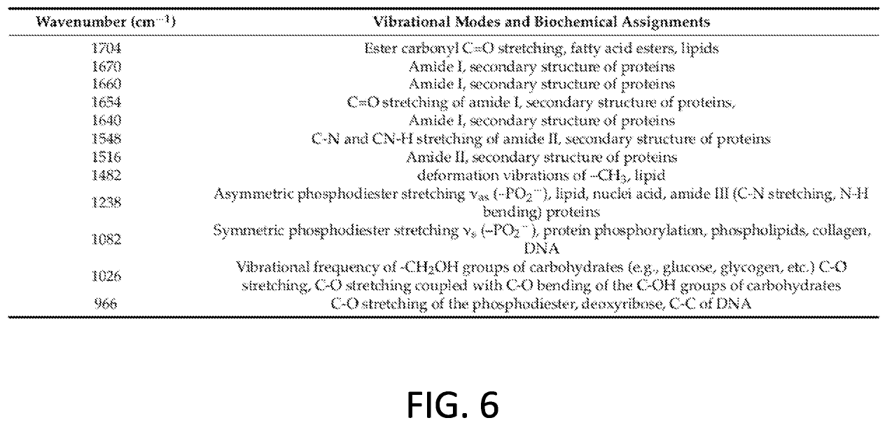

A complete list of spectral assignments is provided in FIG. 6. The Amide I band 210 is herein assigned to a C═O stretching vibration in a peptide backbone structure, and its intensity descends in the order of HK>OED>OSCC. The Amide II band 220 is herein assigned to a bending vibration of a N—H bond and a stretching vibration of a C—N bond in a peptide backbone. The Amide II band 220 shifts toward lower wavenumbers and descends in intensity in the order of HK>OED>OSCC. The Amide III band 230 is herein assigned to N—H bending and C—N stretching vibrations, an asymmetric —PO₂_{^{−}} vibration, and deformational modes of CH₃/CH₂ groups in phospholipids and nucleic acids. The Amide III 230 band shows a descending intensity at 1310 cm⁻¹ in the order of HK>OED>OSCC and an ascending intensity at 1240 cm⁻¹ in the order of OSCC>OED>HK. The glycogen band 240 is herein assigned to stretching vibrations of C—O/C—C groups in a carbohydrate and a symmetric vibration of a —PO₂_{^{−}} group in a phospholipid and/or nucleic acid. The glycogen band 240 declines in intensity in the order of OSCC>OED>HK.

Model Cross-Validation

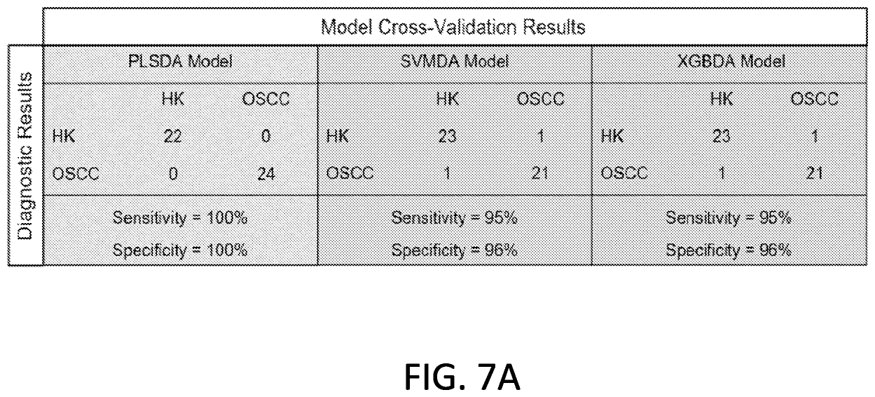

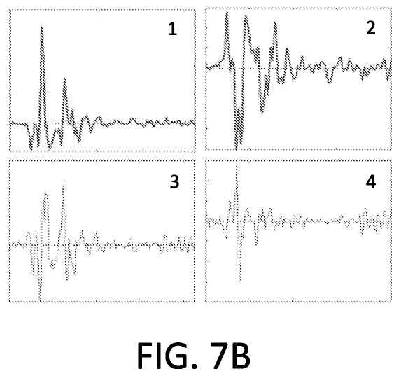

In order to determine which method of supervised discriminatory analysis was best suited for stratification of OED tissues, three such models were applied to spectral data from OSCC and HK tissue samples. Cross-validation results for each of the three models are shown in FIG. 7A. The three models chosen were a PLSDA model, a SVMDA model, and an XGBDA model. 22 representative spectra from 11 tissue samples diagnosed as containing HK cells were analyzed alongside 24 representative spectra from 12 tissue samples diagnosed as containing OSCC cells. As seen in FIG. 5, the PLSDA model showed 100% specificity and sensitivity, correctly separating HK from OSCC tissues.

The four latent variables selected due to the relative success of the PLSDA model were then assessed to determine what spectral features were strongly associated with each latent variable. FIG. 7B shows spectra corresponding to each latent variable in this example, where each of the spectra produced is the result of supplying the machine learning algorithm with 2^{nd} derivatives of spectra derived during hyperspectral imaging. FIG. 7B box 1 shows a spectrum corresponding to a first latent variable, which accounts for 94.50% of variation in the data, shows prominent bands at 1670, 1654, 1548, 1516, 1482, 1238, 1082, 1026, and 966 cm⁻¹. FIG. 7B box 2 shows a spectrum corresponding to a second latent variable, which accounts for 4.48% of variation in the data, shows prominent bands at 1705, 1660, 1640, and 1482 cm⁻¹. FIG. 7B boxes 3 and 4 show spectra corresponding to a third and fourth latent variable, respectively. The third latent variable only accounts for 0.38% of variation in the data, and the fourth latent variable accounts for only 0.29% of variation in the data.

In view of the above, it will be seen that the several objects and advantages of the present invention have been achieved and other advantageous results have been obtained.

As various changes could be made in the above constructions without departing from the scope of the invention, it is intended that all matter contained in the above description or shown in the accompanying drawings shall be interpreted as illustrative and not in a limiting sense.

What is claimed is:

1. A method for stratifying precancerous tissues, said method comprising:
acquiring one or more tissue samples, wherein each tissue sample comprises one or more regions of tissue, further wherein each region of tissue comprises one of a plurality of categories of tissue, wherein the plurality of categories of tissue comprise cancerous tissue, benign tissue, and precancerous tissue,
acquiring a plurality of hyperspectral images of the one or more regions of the one or more tissue samples, wherein the hyperspectral images comprise a plurality of infrared spectra;
performing one or more unsupervised exploratory analyses on the hyperspectral images to generate labeled hyperspectral images;
performing one or more supervised discriminatory analyses on the hyperspectral images of the regions comprising cancerous tissues and the hyperspectral images of the regions comprising benign tissues to generate a discriminatory model;
analyzing the hyperspectral images of the regions comprising precancerous tissues with the discriminatory model to determine whether each of the hyperspectral images of the regions comprising precancerous tissues are most similar to the hyperspectral images of the cancerous tissues or to the hyperspectral images of the benign tissues; and,
assigning the precancerous tissues to a high-risk stratum when the hyperspectral images of the precancerous tissues are most similar to the hyperspectral images of the cancerous tissues, and assigning the precancerous tissues to a low-risk stratum when the hyperspectral images of the precancerous tissues are most similar to the hyperspectral images of the benign tissues.

2. The method of claim 1, wherein the plurality of categories of tissues further comprises one or more categories of intermediate dysplastic tissues, wherein each category of intermediate dysplastic tissues has a set of defining cytological criteria and an associated level of risk of the category of intermediate dysplastic tissue becoming cancerous.

3. The method of claim 1 further comprising assigning the precancerous tissues to one of a plurality of intermediate strata between the ‘low-risk’ stratum and the ‘high-risk’ stratum, wherein each stratum in the of intermediate strata corresponds to one of the categories of intermediate dysplastic tissue.

4. The method of claim 1 further comprising applying one or more image processing steps to the hyperspectral images.

5. The method of claim 4, wherein the one or more image processing steps comprise at least one of conversion between absorbance and transmission data, selection of relevant data regions, digital filtering, light-scattering correction, baseline correction, and normalization.

6. The method of claim 1, wherein the one or more unsupervised exploratory analyses comprise principal components analysis and hierarchical cluster analysis.

7. The method of claim 1, wherein the one or more supervised discriminatory analyses comprise partial least squares discriminant analysis, support vector machines discriminant analysis, and extreme gradient boosting discriminant analysis.

8. A method for stratifying precancerous tissues utilizing a discriminatory model for categorizing each of one or more images of bodily tissues into one of a plurality of categories of tissues, said method comprising:
acquiring a plurality of images of tissues of a tissue sample, each of which correspond to one of the plurality of categories of tissues;
performing one or more unsupervised exploratory analyses on the plurality of images of tissues to generate a plurality of labeled images; and
performing one or more supervised discriminatory analyses on the plurality of labeled images to generate a discriminatory model.

9. The method of claim 8 further comprising applying one or more image processing steps to the plurality of images of tissues.

10. The method of claim 9, wherein the image processing steps comprise at least one of conversion between absorbance and transmission data, selection of relevant data regions, digital filtering, light-scattering correction, baseline correction, and normalization.

11. The method of claim 8, wherein the one or more unsupervised exploratory analyses comprise principal components analysis and hierarchical cluster analysis.

12. The method of claim 8, wherein the one or more supervised discriminatory analyses comprise partial least squares discriminant analysis, support vector machines discriminant analysis, and extreme gradient boosting discriminant analysis.

13. A system for stratifying precancerous tissues in a bodily tissue sample by the risk of the precancerous tissues becoming cancerous utilizing a machine learning algorithm, said system comprising:
one or more tissue sections of the bodily tissue sample comprising at least one section;
a Fourier transform infrared (FTIR) microscope structured and operable to acquire a plurality of hyperspectral images of the at least one section, such that each of the plurality of hyperspectral images is acquired from a region of cancerous tissue or a region of precancerous tissue in the at least one section; and
a computer-based system communicatively linked to the FTIR microscope, the computer-based system structured and operable to execute a machine learning algorithm to:
recognize a plurality of patterns of data in the plurality of hyperspectral images, where the plurality of patterns of data correspond to one or more chemical or biological features of the tissue sample; and,
organize the plurality of hyperspectral images into one of a plurality of categories, wherein each of the plurality of categories corresponds to one or more of the plurality of patterns of data.

14. The system of claim 13, wherein the one or more tissue sections comprises a first section, and further wherein the system further comprises an optical microscope structured and operable to acquire optical image data of the first section of the tissue sample, such that regions of cancerous or precancerous tissue in the first section can be identified.

15. The system of claim 14, wherein the shapes and compositions of the first section and the at least one section are substantially similar, such that the regions of cancerous or precancerous tissue in the first section correspond spatially to the regions of cancerous or precancerous tissue in the second at least one section.

16. The system of claim 13, wherein execution of the machine learning algorithm utilizes statistical methods including supervised discriminatory analyses to organize each of the one or more images of bodily tissues into one of a plurality of categories.

17. The system of claim 13, wherein the plurality of categories comprises categories that correspond to benign, precancerous, and cancerous tissue categories.

18. The system of claim 17, wherein the plurality of categories further comprises multiple distinct precancerous tissue categories.
